(19) United States
(12) Patent Application Publication
(10) Pub. No.: US 2021/0315848 A1
(43) Pub. Date: Oct. 14, 2021

(54) Composition for A Nutraceutical Formulation for Treating Anxiety-Based Disorders

(71) Applicant: Charles Anthony Strongo, SAN CLEMENTE, CA (US)

(72) Inventor: Charles Anthony Strongo, SAN CLEMENTE, CA (US)

(21) Appl. No.: 17/226,049

(22) Filed: Apr. 8, 2021

Related U.S. Application Data
(60) Provisional application No. 63/007,238, filed on Apr. 8, 2020.

(51) Int. Cl. A61K 31/197; A61K 31/14; A61K 31/4415; A61K 36/81; A61K 31/196; A61K 31/4188; A61K 31/51; A61K 31/7004; A61K 31/714; A61K 31/455; A61K 31/525; A61K 33/06; A61K 36/41; A61K 47/46; A61K 31/05; A61K 31/519

(52) U.S. Cl. CPC A61K 31/197; A61K 31/519; A61K 31/4415; A61K 36/81; A61K 31/196; A61K 31/4188; A61K 31/51; A61K 31/7004; A61K 31/714; A61K 31/455; A61K 31/525; A61K 33/06; A61K 36/41; A61K 47/46; A61K 31/05; A61K 31/14

(57) ABSTRACT

A composition for a consumable nutraceutical formulation for treating anxiety-based disorders facilitates reduction of anxiety for persons with Autistic Spectrum Disorders (ASD) or other anxiety-based disorders. The composition includes a quantity of thiamine, a quantity of riboflavin, a quantity of niacin, a quantity of vitamin B6, a quantity of folic acid, a quantity of vitamin B12, a quantity of biotin, a quantity of pantothenic acid, a quantity of choline bitartrate, a quantity of inositol, a quantity of para-aminobenzoic acid (PABA), a quantity of ashwagandha, a quantity of flavor carrier, a quantity of rhodiola, a quantity of gamma aminobutyric acid (GABA), and a quantity of magnesium. The composition is homogeneously mixed into a nutraceutical formulation to be ingested orally or taken by other means.

The current application claims a priority to the U.S. Provisional Patent application Ser. No. 63/007,238 filed on Apr. 8, 2020.

### FIELD OF THE INVENTION

The present invention generally relates to vitamin or supplement formulations. More specifically, the present invention provides a special formulation to help reduce anxiety in various anxiety-based disorders.

### BACKGROUND OF THE INVENTION

An objective of the present invention is to provide a formulation composed of a specialized mixture of Vitamins, Herbs, and Minerals for Autistic Spectrum Disorders (ASD) including, but not limited to, Autism, Fragile X, PTS/PTSD, ADD/ADHD, OD/OCD, depression, and anxiety. The present invention provides a special formula that reduces anxiety and helps people with ASD and other anxiety-based disorders manage their anxiety. The combination of vitamins, minerals, and herbs works on reducing anxiety in the brain, which helps people with any of the anxiety-based disorders.

The formulation of the present invention preferably includes, but is not limited to, Thiamine Mononitrate (Vitamin B-1), Riboflavin (Vitamin B-2), Niacin Amide, Pyridoxine Hydrochloride, Folic Acid, Cyanocobalamin (Vitamin B-12), Biotin, Calcium d-Pantothenate, Choline Bitartrate, Inositol, Para-Aminobenzoic Acid, Ashwagandha, Licorice, Rhodiola, Gamma Aminobutyric Acid (GABA), and Magnesium. The formulation can further include any combination of these vitamins, herbs, or minerals in any quantity or dosage. In addition, the formulation of the present invention can be provided in various forms for oral consumption such as a capsule, patch, gummy, chewable tablet, pill in any shape or form with any flavor or no flavor. A recommended dosage is disclosed below but the recommended dosage can change due to weight.

Additional advantages of the invention will be set forth in part in the description which follows, and in part will be obvious from the description, or may be learned by practice of the invention. Additional advantages of the invention may be realized and attained by means of the instrumentalities and combinations particularly pointed out in the detailed description of the invention section. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

FIG. 1 is a block diagram of the present invention.

FIG. 2 is a block diagram illustrating the proportions of the present invention.

FIG. 3 illustrates the mass percentage composition of an exemplary dose of the formulation of the present invention.

FIG. 4 illustrates the mass composition of the exemplary dose of the formulation of the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising”, when used herein, specify the presence of stated features, steps, operations, elements, various embodiments, and/or components, but do not preclude the presence or addition of one or more other features, steps, operations, elements, various embodiments, components, and/or groups thereof.

Unless otherwise defined, all terms (including technical and scientific terms) used herein have the same meaning as commonly understood by one having ordinary skill in the art to which this invention belongs. It will be further understood that terms, such as those used in commonly used dictionaries, should be interpreted as having a meaning that is consistent with their meaning in the context of the relevant art and the present disclosure and will not be interpreted in an idealized or overly formal sense unless expressly so defined herein.

The present disclosure is to be considered as an exemplification of the invention, and it is not intended to limit the invention to the specific embodiments illustrated by the figures or descriptions.

The present invention is a composition for a nutraceutical formulation for treating anxiety-based disorders. A nutraceutical, also knows as “bioceutical”, is a food product or supplement that may help support health, prevent disease, or provide some other benefit. The formulation of the present invention is specifically designed to reduce anxiety and increase quality of life in people with Autistic Spectrum of Disorders (ASD) and other anxiety-based disorders which affect areas of the brain such as the hypothalamus and hippocampus. This is accomplished through a variety of ingredients known to be helpful in this regard and provided in specific proportions to each other in order to properly produce the desired effect.

In various embodiments of the present invention, the formulation of the present invention can be provided in various forms for oral consumption such as, but not limited to, capsules, gummies, chewable tables, pills of any shape or form, or other forms of consumption. The consumable forms of the formulation may be provided with or without flavoring ingredients. In some embodiments of the present invention, the formulation of the present invention may also be provided in other forms of non-oral consumption, such as, but not limited to, time-delivery patches, suppositories, or injections.

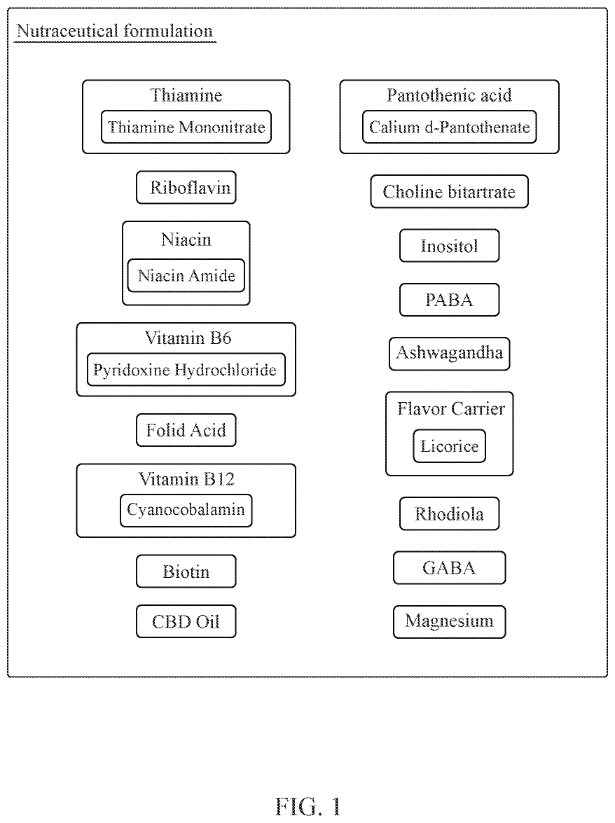

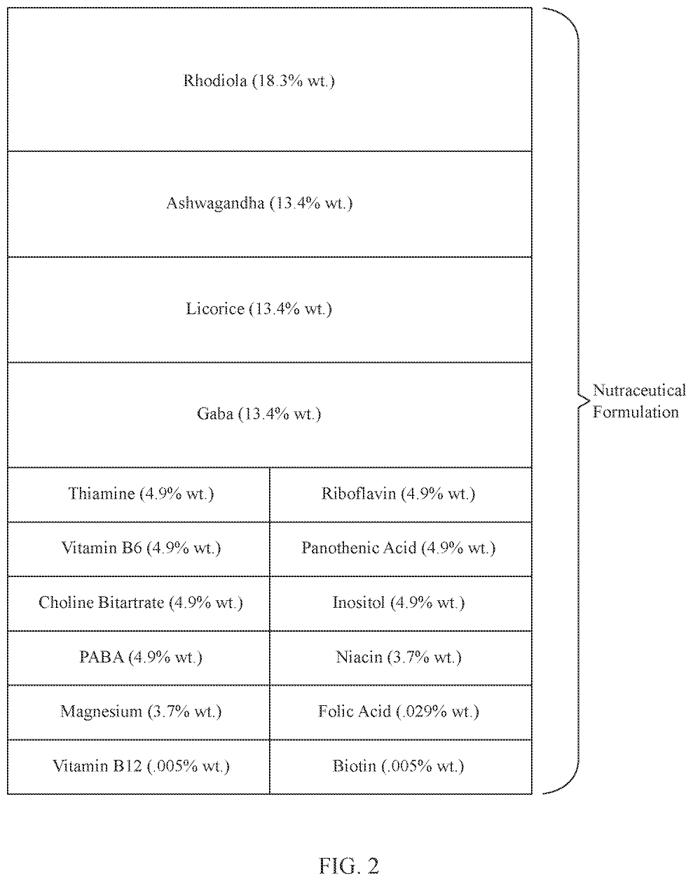

Referring to FIGS. 1-2, a preferred embodiment of the present invention comprises: a quantity of thiamine, a quantity of riboflavin, a quantity of niacin, a quantity of vitamin B6, a quantity of folic acid, a quantity of vitamin B12, a quantity of biotin, a quantity of pantothenic acid, a quantity of choline bitartrate, a quantity of inositol, a quantity of para-aminobenzoic acid (PABA), a quantity of ashwagandha, a quantity of flavor carrier, a quantity of rhodiola, a quantity of gamma aminobutyric acid (GABA), and a quantity of magnesium.

Thus, the quantity of thiamine, the quantity of riboflavin, the quantity of niacin, the quantity of vitamin B6, the quantity of folic acid, the quantity of vitamin B12, the quantity of biotin, the quantity of pantothenic acid, the quantity of choline bitartrate, the quantity of inositol, the quantity of PAPA, the quantity of ashwagandha, the quantity of flavor carrier, the quantity of rhodiola, the quantity of GABA, and the quantity of magnesium are homogeneously mixed, processed, or otherwise combined into a nutraceutical formulation through any suitable known or new manufacturing process. As previously mentioned, the nutraceutical formulation may take the form of one or more pills, capsules, gummies, chewable tablets, candies, food products, or other form suitable for human consumption.

In another preferred embodiment, a quantity of cannabidiol (CBD) oil is further comprised, and the quantity of CBD oil is further homogenously mixed into the nutraceutical formulation.

It should be noted that the constituent ingredients of the present invention are provided specifically in proportion to each other, wherein the proportions disclosed herein are considered necessary for proper effectiveness of the nutraceutical formulation of the present invention in reducing anxiety in a user/consumer of the present invention. Each component should generally not vary from its listed percentage by weight by half a percent in some embodiments, or by one percent in other embodiments. The ratio of each ingredient to each other ingredient should remain approximately the same regardless of the total amount of any given ingredient in the nutraceutical final product.

It is further noted that a recommended treatment dose of the present invention for treatment of anxiety in a user is determined according to bodyweight. Users with higher bodyweight are recommended to ingest proportionally more of the nutraceutical formulation then users with lower bodyweight. For example, a typical dose of the nutraceutical formulation in some embodiments may be equivalent to three capsules. A lighter user may be recommended to take two capsules of the nutraceutical formulation, while a heavier user may be recommended to take the full three capsules of the nutraceutical formulation.

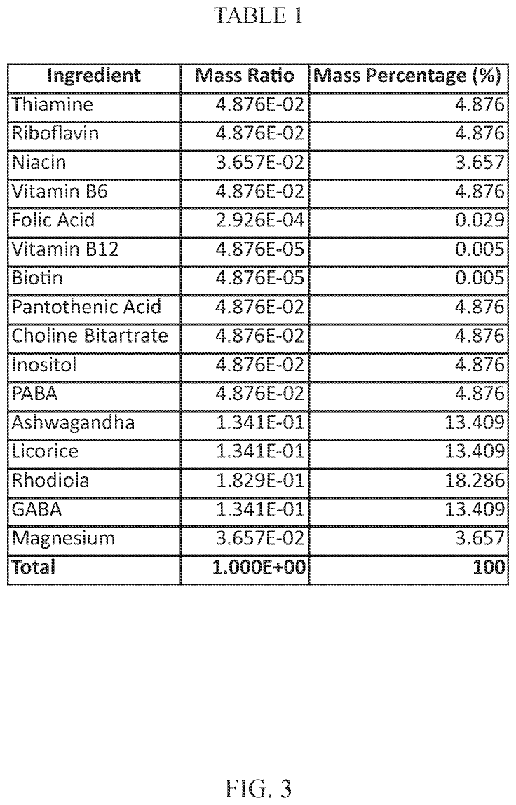

Referring to FIGS. 2-3, in the preferred embodiment, the formulation of the present invention is composed of the aforementioned ingredients according to the following mass percentages. It should be noted for the sake of clarity that any numbers listed herein with multiple places after the decimal should not necessarily imply the need for precision to such a number of decimal places. Moreover, though the weight percentages are listed hereinafter, the proportions of the various ingredients to each other, rather than to the totality of the formulation, is of primary importance in the present invention. Some ingredients may be added, substituted, and removed, potentially resulting in an overall shift in weight percentages, while the proportions of the remaining ingredients to each other remains unchanged.

In the preferred embodiment, the present invention comprises the quantity of thiamine, also known as Vitamin B1, at approximately 4.876 percentage by weight (wt. %) of the nutraceutical formulation. Predetermined amounts of thiamine have been used to successfully treat patients with anxiety disorders. In some embodiments, the thiamine may be provided as thiamine mononitrate, a synthetic stable nitrate salt form of Vitamin B1.

Further, in the preferred embodiment, the present invention comprises the quantity of riboflavin, also known as vitamin B2, at approximately 4.876 wt. % of the nutraceutical formulation.

Further, in the preferred embodiment, the present invention comprises the quantity of niacin at approximately 3.657 wt. % of the nutraceutical formulation. In the preferred embodiment, the niacin is provided as niacin amide, a form of vitamin B3.

Further, in the preferred embodiment, the present invention comprises the quantity of vitamin B6 at approximately 4.876 wt. % of the nutraceutical formulation. The vitamin B6 is preferably provided in the form of pyridoxine hydrochloride.

Further, in the preferred embodiment, the present invention comprises the quantity of folic acid at approximately 0.029 wt. % of the nutraceutical formulation.

Further, in the preferred embodiment, the present invention comprises the quantity of vitamin B12 at approximately 0.005 wt. % of the nutraceutical formulation, preferably in the form of Cyanocobalamin.

Further, in the preferred embodiment, the present invention comprises the quantity of biotin at approximately 0.005 wt. % of the nutraceutical formulation.

Further, in the preferred embodiment, the present invention comprises the quantity of pantothenic acid at approximately 4.876 wt. % of the nutraceutical formulation. The pantothenic acid is preferably provided in the form of calcium d-pantothenate.

Further, in the preferred embodiment, the present invention comprises the quantity of choline bitartrate at approximately 4.876 wt. % of the nutraceutical formulation.

Further, in the preferred embodiment, the present invention comprises the quantity of inositol at approximately 4.876 wt. % of the nutraceutical formulation.

Further, in the preferred embodiment, the present invention comprises the quantity of para-aminobenzoic acid, or PABA, at approximately 4.876 wt. % of the nutraceutical formulation.

Further, in the preferred embodiment, the present invention comprises the quantity of ashwagandha at approximately 13.409 wt. % of the nutraceutical formulation.

Further, in some embodiments, the present invention comprises the quantity of flavor carrier as a quantity of licorice. In such embodiments, the present invention comprises the quantity of licorice at approximately 13.409 wt. % of the nutraceutical formulation. The licorice is provided primarily for flavoring purposes in order to mask any undesirable taste from any of the other ingredients of the present invention. It is contemplated that the licorice may be replaced in other embodiments with other flavoring agents, such as, but not limited to, mint flavoring, or another suitable flavoring agent. It is further contemplated that the weight percentage of the licorice in particular is not considered to be as important to be in proportion with the remaining ingredients of the present invention for the present invention to have the desired effect, and the weight percentage of any other flavoring agent or substance substituted for the licorice in different embodiments may vary as necessary or desired.

Further, in the preferred embodiment, the present invention comprises the quantity of rhodiola at approximately 18.286 wt. % of the nutraceutical formulation.

Further, in the preferred embodiment, the present invention comprises the quantity of gamma-Aminobutyric acid (GABA) at approximately 13.409 wt. % of the nutraceutical formulation.

Further, in the preferred embodiment, the present invention comprises the quantity of magnesium at approximately 3.657 wt. % of the nutraceutical formulation.

In further embodiments, the formulation of the present invention may include additional ingredients to help manage anxiety, such as, but not limited to, CBD oil. In some embodiments, the present invention may further comprise the CBD oil at approximately 0.2438 wt. % of the nutraceutical formulation.

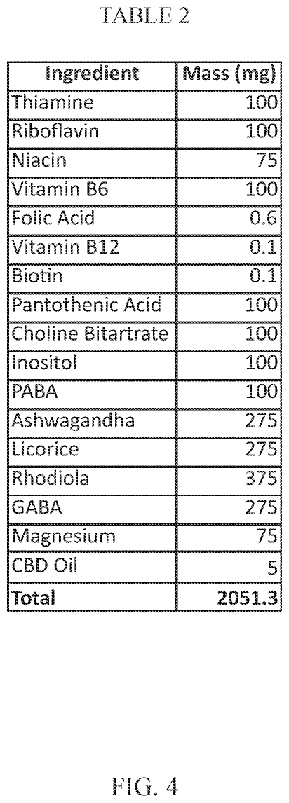

Referring to FIG. 4, in an exemplary embodiment, the formulation of the present invention is provided in a dose with a total mass of 2050.8 milligrams. The exemplary dose of the formulation can be provided in different forms. The formulation of the present invention in the exemplary embodiment includes 100 milligrams of Thiamine, 100 milligrams of Riboflavin, 75 milligrams of Niacin, 100 milligrams of Vitamin B6, 0.6 milligrams of Folic Acid, 0.1 milligrams of Vitamin B12, 0.1 milligrams of Biotin, 100 milligrams of Pantothenic Acid, 100 milligrams of Choline Bitartrate, 100 milligrams of Inositol, 100 milligrams of PABA, 275 milligrams of Ashwagandha, 275 milligrams of Licorice, 375 milligrams of Rhodiola, 275 milligrams of GABA, and 75 milligrams of Magnesium. In a further exemplary embodiment, 5 milligrams of CBD oil may be further provided, resulting in a dose with a total mass of 2051.3 milligrams. The CBD oil should not contain tetrahydrocannabinol, or THC. In alternate embodiments, the formulation of the present invention can be provided in different doses with different masses. Likewise, the ingredients of the formulation of the present invention can include different masses.

Although the invention has been explained in relation to its preferred embodiment, it is to be understood that many other possible modifications and variations can be made without departing from the spirit and scope of the invention as hereinafter claimed.

What is claimed is:

1. A composition for a nutraceutical formulation for treating anxiety-based disorders comprising:
a quantity of thiamine;
a quantity of riboflavin;
a quantity of niacin;
a quantity of vitamin B6;
a quantity of folic acid;
a quantity of vitamin B12;
a quantity of biotin;
a quantity of pantothenic acid;
a quantity of choline bitartrate;
a quantity of inositol;
a quantity of para-aminobenzoic acid (PABA);
a quantity of ashwagandha;
a quantity of flavor carrier;
a quantity of rhodiola;
a quantity of gamma aminobutyric acid (GABA);
a quantity of magnesium; and
the quantity of thiamine, the quantity of riboflavin, the quantity of niacin, the quantity of vitamin B6, the quantity of folic acid, the quantity of vitamin B12, the quantity of biotin, the quantity of pantothenic acid, the quantity of choline bitartrate, the quantity of inositol, the quantity of PAPA, the quantity of ashwagandha, the quantity of flavor carrier, the quantity of rhodiola, the quantity of GABA, and the quantity of magnesium being homogeneously mixed into a nutraceutical formulation.

2. The composition for a consumable nutraceutical formulation for treating anxiety-based disorders as claimed in claim 1 comprising:
a quantity of cannabidiol (CBD) oil;
the quantity of CBD oil being homogenously mixed into the nutraceutical formulation; and
the quantity of CBD oil being approximately 0.2438 percentage by weight (wt. %) of the nutraceutical formulation.

3. The composition for a consumable nutraceutical formulation for treating anxiety-based disorders as claimed in claim 1 comprising:
the quantity of thiamine being approximately 4.876 wt. % of the nutraceutical formulation.

4. The composition for a consumable nutraceutical formulation for treating anxiety-based disorders as claimed in claim 1 comprising:
the quantity of riboflavin being approximately 4.876 wt. % of the nutraceutical formulation.

5. The composition for a consumable nutraceutical formulation for treating anxiety-based disorders as claimed in claim 1 comprising:
the quantity of niacin being approximately 3.657 wt. % of the nutraceutical formulation.

6. The composition for a consumable nutraceutical formulation for treating anxiety-based disorders as claimed in claim 1 comprising:
the quantity of vitamin B6 being approximately 4.876 wt. % of the nutraceutical formulation.

7. The composition for a consumable nutraceutical formulation for treating anxiety-based disorders as claimed in claim 1 comprising:
the quantity of folic acid being approximately 0.029 wt. % of the nutraceutical formulation.

8. The composition for a consumable nutraceutical formulation for treating anxiety-based disorders as claimed in claim 1 comprising:
the quantity of vitamin B12 being approximately 0.005 wt. % of the nutraceutical formulation.

9. The composition for a consumable nutraceutical formulation for treating anxiety-based disorders as claimed in claim 1 comprising:
the quantity of biotin being approximately 0.005 wt. % of the nutraceutical formulation.

10. The composition for a consumable nutraceutical formulation for treating anxiety-based disorders as claimed in claim 1 comprising:
the quantity of pantothenic acid being approximately 4.876 wt. % of the nutraceutical formulation.

11. The composition for a consumable nutraceutical formulation for treating anxiety-based disorders as claimed in claim 1 comprising:
the quantity of choline bitartrate being approximately 4.876 wt. % of the nutraceutical formulation.

12. The composition for a consumable nutraceutical formulation for treating anxiety-based disorders as claimed in claim 1 comprising:
the quantity of inositol being approximately 4.876 wt. % of the nutraceutical formulation.

13. The composition for a consumable nutraceutical formulation for treating anxiety-based disorders as claimed in claim 1 comprising:
the quantity of PABA being approximately 4.876 wt. % of the nutraceutical formulation.

14. The composition for a consumable nutraceutical formulation for treating anxiety-based disorders as claimed in claim 1 comprising:
the quantity of ashwagandha being approximately 13.409 wt. % of the nutraceutical formulation.

15. The composition for a consumable nutraceutical formulation for treating anxiety-based disorders as claimed in claim 1 comprising:
the quantity of flavor carrier being a quantity of licorice.

16. The composition for a consumable nutraceutical formulation for treating anxiety-based disorders as claimed in claim 15 comprising:
the quantity of licorice being approximately 13.409 wt. % of the nutraceutical formulation.

17. The composition for a consumable nutraceutical formulation for treating anxiety-based disorders as claimed in claim 1 comprising:
the quantity of rhodiola being approximately 18.286 wt. % of the nutraceutical formulation.

18. The composition for a consumable nutraceutical formulation for treating anxiety-based disorders as claimed in claim 1 comprising:
the quantity of GABA being approximately 13.409 wt. % of the nutraceutical formulation.

19. The composition for a consumable nutraceutical formulation for treating anxiety-based disorders as claimed in claim 1 comprising:
the quantity of magnesium being approximately 3.657 wt. % of the nutraceutical formulation.
